(19) United States
(12) Patent Application Publication
(10) Pub. No.: US 2007/0148159 A1
(43) Pub. Date: Jun. 28, 2007

(54) Use of crotoxin as an analgesic - CIP

(76) Inventors: Paul F. Reid, Plantation, FL (US); Zheng Hong Qin, Malden, MA (US)

Correspondence Address: Robert J. Van Der Wall;Gables One Tower, Suite #1275, 1320 South Dixie Highway, Coral Gables, FL, 33146, US

(21) Appl. No.: 11/313,377

(22) Filed: Dec. 22, 2005

(51) Int. Cl. A61K 38/48; A61K 31/60

(52) U.S. Cl. 424/94.64; 514/165

(57) ABSTRACT

A method of producing analgesia and enhancing analgesia is disclosed. The method includes administering a beta-neurotoxin that is characterized by its ability to bind to pre- and postsynaptic receptors resulting in the inhibition of neurotransmission.

### BACKGROUND OF THE INVENTION

1. Field of the Invention

The present invention relates to a class of proteins and a method for the treatment of chronic pain, especially to the treatment of heretofore intractable pain as associated with advanced cancer. The pain associated with neurological conditions, rheumatoid arthritis, viral infections and lesions also responds to treatment with the present invention. The composition consists of a beta-neurotoxin in acceptable carrier for either parenteral or topical administration.

2. Description of the Prior Art

Numerous natural products have demonstrated analgesic activity in addition to neurotoxins comprising chemical and biological agents. In fact the most potent neurological toxin, botulinum, has been shown to possess useful analgesic properties as have the smaller peptides, and Conotoxins, from the venom of Conus snails. Snake venoms have been used as analgesics and subsequently peptides and neurotoxins from those venoms have been isolated and identified as having analgesic potential. The neurotoxins, cobrotoxin and crotamine, from cobras and rattlesnakes respectively, have each been assayed in animal models of pain and were found to have what was considered to be potent activity in comparison to opiates (Chen and Robinson, 1990, Mancin et al., 1998).

Phospholipases A₂ are conspicuous components of snake venoms. These enzymes are compact globular proteins with molecular weight of about 14000 kD as monomers or are found as multimeric complexes, and exhibit a high degree of similarity in amino acid sequences, secondary and tertiary structure (Verheij et al., 1981). They catalyze the stereospecific, Ca²⁺-dependent hydrolysis of the fatty acyl ester bond in position 2 of all common 3-sn-phosphoglycerides (plasmalogens or glyceryl ethers) resulting in free fatty acid and the 1-acyl phosphoglyceride or lysophospholipid (van Deenen & De Haas, 1963). At difference with the highly conserved (invariant) amino acid residues responsible for their catalytic activity (i.e., the catalytic network), the structural elements responsible for the wide variety of pharmacological effects exhibited by this large family of homologous proteins are presently unknown (Kini and Evans, 1989; Kini, 1997). In spite of their similarities in sequence, secondary and tertiary structures, these enzymes may induce one or more pharmacologic effects (i.e., neurotoxic, myotoxic, cardiotoxic, anticoagulant, hemolytic, edema-inducing activities, etc.), but not all the effects are exhibited by all phospholipases A₂. They have also been associated with the induction of pain through inflammation in cases of arthritis. Beta—neurotoxins are characterized by their intrinsic phospholipase A₂ activity that ultimately causes the breakdown of neurotransmitter release from presynaptic neurons.

Crotoxin, a beta neurotoxin from Crotalus durissus terrificus (South American rattlesnake) venom displays cytotoxic activity in vitro against a number of murine and human tumor cell lines. Although not fully understood, Crotoxin's anticancer mechanism involves the recognition of a set of structural elements present in the membrane of the target cells, which results in binding of the toxin. Structural perturbation of the membrane resulting from anchoring of the toxin and subsequent hydrolysis of membrane phospholipids leads to cell death. The possibility of achieving cytotoxicity in malignant cells by means of the specific binding of a phospholipase A₂ and subsequent phospholipid hydrolysis constitute a novel approach to cancer therapy. This activity is distinct from the protein's neurotoxic capacity.

Other references of interest include several patents, Haast, U.S. Pat. No. 4,341,762; Plata et al., U.S. Pat. No. 5,164,196 and Vidal, U.S. Pat. No. 5,232,911; Lipps, U.S. Pat. Nos. 5,565,431 and 6,307,031; and Aoki, U.S. Pat. No. 6,869,610. Literature references of interest are: Barral-Netto M, von Sohsten R L, Toxicon. (1991) 29(4-5):527-31; Bon C, Bouchier C, Choumet V, Faure G, Jiang M S, Lambezat M P, Radvanyi F, Saliou B; Acta Physiol Pharmacol Lainoam (1989) 39:439-448; Brazil O V, Fontana M D, Heluany N F. Nat Toxins. (2000) 9(1):33-42; Breithaupt, H. Naunyn-Schmiedeberg's Arch. Pharmacol. (1976) 292, 271-298; Cardi B A, Andrade H F Jr, Rogero J R, Nascimento N. Nat Toxins. (1998) 6(1):19-25; Cardoso D F, Mota I; Toxicon (1997) 35: 607-612; Cardoso D F, Lopes-Ferreira M, Faquim-Mauro E L, Macedo M S, Farsky S H; Mediators Inflamm (2001) 10: 125-133; Chaim-Matyas A, Borkow G. Ovadia M., Biochem Int. (1991) 24(3):415-21; Chang, C. C.; Lee, C. Y. Naunyn-Schmiedeberg's Arch. Pharmacol. (1977) 296, 159-168; Chiou S H, Raynor R L, Zheng B, Chambers T C, Kuo J F. Biochemistry. (1993) 2; 32(8), 2062-7; Choumet V, Lafaye P. Mazie J C, Bon C; Biol Chem (1998) 379:899-906; Corin, R. E.; Viskatis, L. J.; Vidal, J. C., Etcheverry, M. A. (1993). Invest. New Drugs 11, 11-15; Costa L A, Miles F, Diez R A, Araujo C E, Coni Molina C M and Cervellino J C; Anticancer Drugs 8 (9), 829-34 (1997) Delot, E., & Bon, C. (1992); J. Neurochem. 58, 311-319; Delot E, Bon C; Biochemistry (1993) 32:10708-10713; De Tolla, L. J.; Stump, K. C.; Russell, R.; Viskatis, L. J.; Vidal, J. C.; Newman, R. A.; Etcheverry, M. A. Toxicology (1995) 99, 31-46; Donato, N. J.; Martin, C. A.; Perez, M.; Newman, R. A.; Vidal, J. C.; Etcheverry, M. A. Biochem. Pharmacol. (1996) 51, 1535-1543; Donato, N. J., Yan, D. H., Hung, M. C., Rosenblum, M. G. (1996) Cell Growth Differ. (1996) 4, 411-419; Fletcher J E, Jiang M S. Toxicon. (1993) 31(6):669-95; Freitas T V, Fortes-Dias C L, Diniz C R Toxicon. (1990) 28(12):1491-6; Habermann, E.; Walsch, P.; Breithaupt, H. Naunyn-Schmiedeberg's Arch. Pharmacol. (1972) 273, 313-330; Hawgood, B.; Bon, C. (1991): Snake venom presynaptic toxins. In Handbook of Natural Toxins, Vol. 5. Reptile and Amphibian Venoms. Ed. A. T. Tu, pp 3-52. Marcel Dekker, New York; Hirai, M., Gamou, S., Minoshima, S., Shimizu, N. J. Cell Biol. (1988) 107, 791-799; Gopalakrishnakone P, Hawgood B J. Toxicon. (1984) 22(5):791-804; Hinman C L, Lepisto E, Stevens R, Montgomery I N, Rauch H C, Hudson R A. Toxicon. (1987) 25(9):1011-4; Holzer, M and MacKessy, S. P., Toxicon, (1996) 34, No. 10, 1149-1155; Hseu M J, Yen C H, Tzeng M C. FEBS Lett. (1999) 26, 445(2-3):440-4; Kattah L R, Ferraz V, Matos Santoro M, Ribeiro da Silva Camargos E, Ribeiro Diniz C, De Lima M E; Toxicon (2002) 40:43-49; Leung W W, Keung W M, Kong Y C. Naunyn Schmiedebergs Arch Pharmacol. (1976) 25, 292(2):193-8; Martins A M, Toyama M H, Havt A, Novello J C, Marangoni S. Fonteles M C, Monteiro H S. Toxicon, (2002) 40 (8), 1165-1171; Mebs D, Ownby C L. Pharmacol Ther. (1990) 48 (2):223-36; Miyabara E H, Tostes R C, Selistre de Araujo H S, Aoki M S, Salvini T F, Moriscot A S. Toxicon. (2004) 43(1):35-42; Monteiro H S, da Silva I M, Martins A M, Fonteles M C. Braz J Med Biol Res. (2001) 34 (10), 1347-1352; Nakazone A K, Rogero J R, Goncalves J M. Braz J Med Biol Res. (1984) 17(2):119-28; Newman, R. A.; Vidal, J. C.; Viskatis, L. J.; Johnson, J. I.; Etcheverry, Invest. New Drugs (1993) 11, 151-159; Newman, R. A.; Yu, Y. H.; Xu, F. J.; Thonton, A.; Bast, R. C.; Von Hoff, D. D.; Vidal, J. C.; Etcheverry, M. A., Proc. Amer. Assoc. Cancer Res. (1996) 37, 393; Nishikawa, K., Rotbein, J., Wijjeswarapu, D., Owen-Schaub, L., Rosenblum, M. G., Donato, N. J., Lymphokine Cytokine Res. (1994) 13, 37-45; Okamoto, M.; Viskatis, L. J.; De la Roza, G.; Vidal, J. C., J. Pharmacol. Exp. Ther. (1993) 265, 41-46; Ownby, C. L.; Selistre de Araujo, H. S.; White, S. P.; Fletcher, J. E., Toxicon (1999) 37, 411-445; Ownby CL, Fletcher JE, Colberg T R. Toxicon. (1993) 31 (6):697-709; Paull, K. D., Shoemaker, R. H., Hodes, L., Monks, A., Scudeiro, D. A., Rubinstein, L., Plowman, J., Boyd, M. R., J. Natl. Cancer Inst. (1989) 81, 1088-1092; Ollivier-Bousquet M, Radvanyi F, Bon C., Mol Cell Endocrinol. (1991) 82 (1):41-50; Rangel-Santos AC, Mota I; Toxicon (2000) 38:1451-1457; Rudd, C. J.; Viskatis, L. J.; Vidal, J. C.; Etcheverry, M. A., Invest. New Drugs (1994) 12, 183-184; Shin D M, Donato N J, Perez-Soler R. Shin H J C, Wu J Y, Zhang P, Lawhorn K, Khuri F R, Glisson B S, Myers J, Clayman G, Pfister D, Falcay J, Waksal H, Mendelsohn J and Hong W K, Clin. Can Res., (2001) 7, 1204-13; Simpson L L, Lautenslager G T, Kaiser I I, Middlebrook J L., Toxicon. (1993) 31(1):13-26; Sribar J. Copic A, Paris A, Sherman NE, Gubensek F. Fox J W, Krizaj I., J Biol Chem. (2001), 276(16):12493-6; Stanchi N O, Arias D, Bartolucci E, Martino P E, Gimeno E J, Diez R A, Costa L A. Arzneimittelforschung., (2000), 50 (9), 862-866; Stanchi N O, Arias D, Martino P E, Diez R A, Costa L A. Farmaco. (2002), 57(2):167-70; Strong, P. N. (1987) Presynaptic phospholipase A₂ neurotoxins. Relationship between biochemical and electrophysiological approaches to the mechanism of toxic action. In: The Cellular and Molecular Basis of Cholinergic Function (Dowdall, M. J. and Hawthorne J. N., eds)., pp 534-549. Ellis Horwood, Chichester.; Tzeng M C, Yen C H, Hseu M J, Tseng C C, Tsai M D, Dupureur C M. Toxicon. 1995 Apr;33(4):451-7; Vernon L P, Rogers A. Toxicon. (1992) 30 (7):711-21; Vital Brazil, O., Mem. Inst. Butantan (1966) 33, 981-992; Zhang Y, Tu A T. Neurotoxicology. (2002) 23 (3):273-9; Zheng B, Chambers T C, Raynor R L, Markham P N, Gebel H M, Vogler W R, Kuo J F. J Biol Chem. (1994) 22, 269(16):12332-8;Tsiang H., de la Porte S., Ambroise D. J., Derer M. And Koenig J.; J. Neuropathol. Exp. Neurol. 45: 28-42; Tu A. T.; Ann. Rev. Biochem. 42:235-258(1973); Carstens E, Anderson K A, Simons C T, Carstens M I, Jinks S L. Psychopharmacology (Berl) 2001 Aug;157(1):40-5 “Analgesia induced by chronic nicotine infusion in rats: differences by gender and pain test.”; Damaj, M. I., Fei-Yin, M., Dukat, M., Glassco, W., Glennon, R. A. and Martin, B. R., JPET 1998 284:1058-1065, “Antinociceptive Responses to Nicotinic Acetylcholine Receptor Ligands after Systemic and Intrathecal Administration in Mice.”; Damaj M I, Meyer E M, Martin B R. Neuropharmacology 2000 Oct;39(13):2785-91 “The antinociceptive effects of alpha7 nicotinic agonists in an acute pain model.”; Decker M W, Meyer M D, Sullivan J P. Expert Opin Investig Drugs 2001 Oct;10(10):1819-30 “The therapeutic potential of nicotinic acetylcholine receptor agonists for pain control.”; *Irnaten M, Wang J, Venkatesan P, Evans C, K Chang K S, Andresen M C, Mendelowitz D. Anesthesiology 2002 Mar;96(3):667-74 “Ketamine inhibits presynaptic and postsynaptic nicotinic excitation of identified cardiac parasympathetic neurons in nucleus ambiguus.”; Lieb K, Treffurth Y, Berger M, Fiebich B L. Neuropsychobiology 2002;45 Suppl 1:2-6 “Substance P and affective disorders: new treatment opportunities by neurokinin 1 receptor antagonists?”; Min C K, Owens J. Weiland G A. Mol Pharmacol 1994 Feb;45(2):221-7 “Characterization of the binding of [3H]substance P to the nicotinic acetylcholine receptor of Torpedo electroplaque.”; Schaible H G, Ebersberger A, Von Banchet G S. Ann N Y Acad Sci 2002 Jun;966:343-354 “Mechanisms of Pain in Arthritis.”; Schmidt B L, Tambeli C H, Gear R W, Levine J D. Neuroscience 2001;106(1):129-36 “Nicotine withdrawal hyperalgesia and opioid-mediated analgesia depend on nicotine receptors in nucleus accumbens.”; *Shiraishi M, Minami K, Uezono Y. Yanagihara N, Shigematsu A, Shibuya I. Br J Pharmacol 2002 May;136(2):207-16 “Inhibitory effects of tramadol on nicotinic acetylcholine receptors in adrenal chromaffin cells and in Xenopus oocytes expressing alpha7 receptors.”

### SUMMARY OF THE INVENTION

It is a principal object of the present invention to provide a method for treating pain associated with advanced cancer, neuropathy, painful viral infections and their lesions in addition to rheumatic pain.

It is a further object of the present invention to provide a composition of matter and therapy for the treatment of pain of the aforementioned type, whose composition and therapy are safe, effective and may be administered over long periods of time.

It is another object of the present invention to provide a composition of matter and method of therapy for the treatment of pain apart and separate from the treatment of the diseases which cause the pain.

Other objects and advantages will be apparent to those skilled in the art from the following disclosures and the appended claims.

The present invention accomplishes the above-stated objectives, as well as others, as may be determined by a fair reading and interpretation of the entire specification

### DETAILED DESCRIPTION OF THE INVENTION

As required, detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the claims to be later appended and as a representative basis for teaching one skilled in the art to variously employ the present invention in virtually any appropriate circumstance.

In general, brain pathways governing the perception of pain are still incompletely understood. Sensory afferent synaptic connections to the spinal cord, termed “nociceptive pathways” have been documented in some detail. In the first leg of such pathways, C- and A-fibers which project from peripheral sites to the spinal cord carry nociceptive signals. Poly-synaptic junctions in the dorsal horn of the spinal cord are involved in the relay and modulation of sensations of pain to various regions of the brain, including the periaqueductal grey region (McGeer). Analgesia, or the reduction of pain perception, can be affected directly by decreasing transmission along such nociceptive pathways. Analgesic opiates are thought to act by mimicking the effects of endorphin or enkephalin peptide-containing neurons, which synapse pre-synaptically at the C- or A-fiber terminal and which, when they fire, inhibit release of glutamate and substance P. The key transmitter is glutamate that activates N-methyl-d-aspartate (NMDA) and non-NMDA receptors on spinal cord neurons. Substance P (SP) is a neuropeptide which is abundant in the periphery and the central nervous system, where it is co-localized with other neurotransmitters such as serotonin or dopamine. SP has been proposed to play a role in the regulation of pain including migraine and fibromyalgia, asthma, inflammatory bowel disease, emesis, psoriasis as well as in central nervous system disorders.

Chronic or intractable pain, such as may occur in conditions such as degenerative bone diseases and cancer, is a debilitating condition which is treated with a variety of analgesic agents, and often opioid compounds such as morphine. The synthesis of analgesics, particularly of morphine-like compounds, has always been a point of major interest in drug research. For decades, scientists throughout the world have attempted to develop effective analgesics by “re-building” the morphine molecule, considering its constitution a combination of certain “basic skeletons” from which they started their syntheses. Meperidine hydrochloride (also known as Dolantin or Demerol) is one such synthetic narcotic analgesic. It is one-tenth as potent an analgesic as morphine and its analgesic effect is halved again when given orally rather than parenterally. The onset of activity occurs within 10-45 minutes with a duration of 2-4 hours. It has superseded morphine as the preferred analgesic for moderate to severe pain. It has been found to be particularly useful for minor surgery, as in orthopedics, ophthalmology, rhinology, laryngology, and dentistry. It is also used in parenteral form for preoperative medication, adjunct to anesthesia and obstetrical analgesia. Like morphine, its binding to opioid receptors produces both psychologic and physical dependence with overdosing causing severe respiratory depression in addition to a number of other undesirable side effects and drug interactions.

Although calcium blocking agents, including a number of L-type calcium channel antagonists, have been tested as adjunct therapy to morphine analgesia, positive results are attributed to direct effects on calcium availability, since calcium itself is known to attenuate the analgesic effects of certain opioid compounds (Ben-Sreti). EGTA, a calcium chelating agent, is effective in increasing the analgesic effects of opioids. Moreover, in some cases, results from tests of calcium antagonists as adjunct therapy to opioids have been contradictory; some L-type calcium channel antagonists have been shown to increase the effects of opioids, while others of these compounds have been shown to decrease opioid effects (Contreras). A conotoxin, SNX111, that inhibits calcium channels has been developed as an analgesic though it must be administered intrathecally for optimum effect.

Due to the limitations of such analgesics, a number of novel alternatives are currently under investigation, including neuronal nicotinic acetylcholine receptor (NAChR) agonists. It has been found that the acute administration of nicotine induces analgesia with subsequent development of tolerance. Interestingly, in nicotine-naive rats, injection of the nicotinic receptor antagonist mecamylamine into the nucleus accumbens (where the site for activity of substances of abuse such as opioids has been implicated in pain modulation) blocked antinociception produced by either systemic morphine, intra-accumbens co-administration of a mu- and a delta-opioid receptor agonist, or noxious stimulation (i.e., subdermal capsaicin in the hindpaw). Intra-accumbens mecamylamine by itself precipitated significant hyperalgesia in nicotine-tolerant rats which could be suppressed by noxious stimulation as well as by morphine. Thus, nicotinic receptors have been found to play a role in modulating pain transmission in the CNS. Activation of other cholinergic pathways by nicotine and nicotinic agonists has been shown to elicit antinociceptive effects in a variety of species and pain tests.

However, in an apparent contradiction to the above, nicotinic antagonists may also have a role in pain relief. Tramadol and Ketamine have been used clinically as analgesics. However, until recently, their mechanism of analgesic effect was unknown. Studies showed that Tramadol inhibited nicotinic currents carried by alpha7 receptors expressed in Xenopus oocytes (Shiraishi et al.). It also inhibited both alpha-bungarotoxin-sensitive and -insensitive nicotinic currents in bovine adrenal chromaffin cells. It was concluded that tramadol inhibited catecholamine secretion partly by inhibiting nicotinic AChR functions. The alpha7 subtype was one of those inhibited by Tramadol. Ketamine was found to inhibit the nicotine-evoked presynaptic facilitation of glutamate release (Irnaten et al.). Oddly, alpha-bungarotoxin, an antagonist of alpha7 containing nicotinic presynaptic receptors, blocked specific Ketamine actions. It was concluded that Ketamine inhibits the presynaptic nicotinic receptors responsible for facilitating neurotransmitter release, as well as the direct ligand-gated inward current.

Crude cobra or rattlesnake venoms have proven to have analgesic properties. Obviously, at the time crude venoms were employed without even an adequate knowledge of the source or mechanism. Sometimes venoms from cobras captured in India or South Africa were employed indistinctly (See Haast, U.S. Pat. No. 4,341,762). A number of neurotoxins from venoms have demonstrated antinociceptive properties such as the aforementioned conotoxin SNX111, crotamine and cobrotoxin. Herein is the newly described analgesic activity of Crotoxin. Crotoxin as a treatment for malignancies has beer previously described and successful therapy was associated with the amelioration of pain prompting further investigation into this effect. The use of Crotoxin solely to relieve pain has not been previously described.

Crotoxin is a 24 kD non covalent complex formed by two non identical subunits: a basic one (crotoxin subunit B, 14.5 kD) and an acidic one (crotoxin subunit A, 9.5 kD). Crotoxin subunit A is non-toxic and devoid of catalytic activity. It is formed by three polypeptide chains cross-linked by seven disulfide bonds. When properly aligned, the polypeptide chains (A, B and C) exhibit sequence similarities with other non-toxic phospholipases. Isoforms of subunit A which differ in two or three amino acid residues at the beginning and at the end of chain A appear to be generated by the proteolytic cleavage of a precursor polypeptide homologous to a phospholipase A₂ (Bon, 1997). The structure and production is described in detail and incorporated here by way of reference (Plata et al., U.S. Pat. No. 5,164,196 and Vidal, U.S. Pat. No. 5,232,911). The resulting neurotoxin solution, i.e., crotoxin, is filter sterilized to remove residual bacteria. The solution needs to be diluted prior to filling and administration usually to between 500 and 2000 mcg/ml. Any suitable preservative for parenteral administration can be employed such as methyl paraben, benzalkonium chloride or metacreosol.

Crotoxin, beta-Bungarotoxin and Taipoxin are a few of a group of neurotoxic phospholipases A2 (beta-neurotoxin) capable of affecting the presynaptic activity to bring about ultimate blockade of synaptic transmission. It has previously shown that iodinated crotoxin and taipoxin bind specifically with high affinity to the isolated synaptic membrane fraction from guinea-pig brain, whereas specific binding is not detected with the nontoxic pancreatic phospholipase A2. Crotoxin impairs the release of acetylcholine at neuromuscular junctions, primarily at the presynaptic level. The enzymatic activity is considered to be necessary but not solely sufficient for the blockade (Tzeng et al, 1995). Since many phospholipases A2 with comparable or even higher enzymatic activity are not toxic, it has been postulated that the difference in potency lies in the specific binding affinity of Crotoxin to the presynaptic membrane, imparted by the A subunit.

Experiments based on photoaffinity labeling and simple chemical cross-linking techniques have led to the identification of three polypeptides preferentially present in neuronal membranes as subunits of the binding protein(s) for crotoxin and/or taipoxin. Some other toxic phospholipases A2 also appear to be ligands for the three polypeptides. It has been found that under Ca(2+)-free condition, taipoxin or crotoxin inhibit with an IC50 of 20-1000 nM the Na(+)-dependent uptake of norepinephrine, dopamine and serotonin by synaptosomes. In contrast, choline uptake is not affected (Tzeng et al, 1995).

Crotoxin is also known to desensitize the nicotinic receptor of Torpedo marmorata and Electrophorus electricus electroplaques providing it with two potential mechanisms of action. It has been found that the purely cholinergic synaptosomes from the Torpedo electric organ provided a convenient model to study the pharmacology of crotoxin and other related neurotoxins (Delot, E., & Bon, C., 1992). Labeled ¹²⁵I crotoxin demonstrated saturable binding to Torpedo presynaptic membranes. In the range of concentrations that was effective on synaptosomes, crotoxin bound to a single class of sites without cooperativity. 4-Aminopyridine, a voltage gated potassium channel antagonist, inhibits the crotoxin-induced blockade of the end-plate depolarization produced by carbachol showing that the postsynaptic effect of crotoxin at the guinea-pig muscle end-plate also results from nicotinic receptor desensitization.

It is interesting to note that the venom of Crotalus durissus terrificus (CDT) venom has been utilized for decades as an analgesic often being administered orally. Several investigations were undertaken to examine which components of the venom were associated with this effect. Georgi et al (1993)reported that the subcutaneous (s.c.), intraperitoneal (i.p.) or oral (p.o.) administration of CDT venom caused an antinociceptive effect in mice as measured by the acetic acid-induced writhing method and the hot plate test. The antinociceptive activity was dose and time dependent and persisted after neutralization of the venom with a specific antivenom. It was demonstrated that the factor(s) had an apparent molecular weight of less than 3000 and that its antinociceptive effect was abolished by trypsin treatment suggesting it was a small peptide. Morphine enhanced the analgesic effect of CDT venom and naloxone antagonized the effect suggesting an endorphin-like activity for the identified factor(s) (Piccolo et al., 2000). Mancin et al (1998) described the analgesic effect of crotamine, a significant neurotoxic component of the venom.

In the effort to examine Crotoxin's potential as an anticancer product human clinical trials were conducted. Several trial participants experienced significant reduction in their cancer burden and consistently reported pain relief supported by a reduction in the consumption of concomitant analgesics (Costa et al., 1997, Cura et al., 2001). From these observation several conclusions were possible. The bioburden or accumulation of the tumor mass was causing the pain and that the action of Crotoxin in reducing the bulk of the tumor permitted a reduction in perception of pain by the subject. In the same article Cura et al (2001) expand on the pain observation citing a single case in which a reduction in pain was noted in spite of disease progression. It was reported that a clear explanation for this observation was not available. The use of concurrent pain killer use prevented the certain conclusion that Crotoxin had analgesic properties and Crotoxin solely as an analgesic was not anticipated or rendered obvious by either Costa or Cura. These observations suggested at best that Crotoxin may be able to enhance the activity of co-administered analgesics. It was this observation that prompted the contemplation that Crotoxin had independent analgesic activity distinct from its antitumor capabilities. That is an important distinction with the prior art. It was therefore decided to reduce this idea to practice by devising and conducting research in animal models of pain and in the absence of cancerous lesions. The positive results obtained in animal models permits the discovery to be employed outside the scope of advanced cancer and into other chronic pain afflictions associated with other disease states. Crotoxin has not been employed to control pain in any other disease state nor was it expressly employed to control pain in cancer subjects whose tumors failed to respond to the antitumor action of the protein.

Crotoxin was compared to Dolantin for its ability to delay the hot-plate response in the hot-plate model. In comparison to control animals, animals treated with Dolantin showed a rapid onset with a maximal effect noted at 30 minutes. At 90 minutes, the effect of Dolantin was wearing off reducing further at 120 minutes. The effect of crotoxin was slower at onset though ultimately achieving an almost equivalent effect to Dolantin. Crotoxin's effect continued to increase with a maximal effect at 180 minutes (the test's end point). The data suggested that crotoxin had an activity equivalent in potency to Dolantin but with slower onset and a more prolonged effect. However, over 400 times more Dolantin was administered to the subject animals relative to the quantity of Crotoxin. On a molar basis the difference is even more acute. Dolantin's molar equivalent with a molecular weight (M_{w}) of 283.8 was 0.140 millimoles in comparison to crotoxin's Mw, at a minimum of 21,000 was 0.004 micromoles, a 35,000 fold difference.

The data further suggested that the combination of both drug could be beneficial giving the rapid onset of Dolantin and the prolonged effect of the Crotoxin. Further benefits would be reduced dependence on a known addictive drug with significant side effects.

Naloxone, an opiate inhibitor and Atropine, a muscarinic ligand failed to inhibit the activity of Crotoxin and provided evidence that opiate receptors and muscarinic acetylcholine receptors were uninvolved in the amelerioration of pain. The reported dual mechanism of crotoxin suggests that the anti-nociceptive effect could result from the impairment of acetylcholine or glutamate release from the presynaptic surface and nicotinic acetylcholine receptor desensitization. Desensitization of the nicotinic receptor would mimic the analgesic effects described for nicotine. While nicotine is an agonist of it respective receptor it is the resulting desensitization of the receptor that is now believed form the basis of its antinociceptive effects. It would also be consistent with the analgesic measurements made for the nicotinic antagonist cobrotoxin by Chen and Robinson (1990).

In order to ensure that the apparent analgesic activity was not due to neurotoxin-induced paralysis locomotor activity in mice was measured using an Animes Type S activity meter (LKB, Farad, Sweden) with the setting at maximum sensitivity. Every movement of the mice was recorded automatically by the instrument. The locomotor activity of mice was then observed for 1-3 h 15 min after intraperitoneal (i.p.) injection of Crotoxin. It was confirmed by comparison to controls that the antinociceptive effect of Crotoxin was not due to a neuromuscular or paralytic effects since the doses that induced significant antinociceptive effects did not inhibit locomotor activity. In contract to the conotoxins, it was evident that the peripheral administration of the drug was efficacious.

In the treatment of pain the administration of crotoxin is required regularly, at least once every other day extending to several applications daily. Parenteral (intravenous, intramuscular or subcutaneous) neurotoxins should deliver at least 10 mcg/day up to a maximum of 3 mg. Studies have suggested the average dose to be between 100 and 1000 mcg/day for purified neurotoxin preparations. Higher doses can be employed for more rapid onset of effect with the preferred route being intravenous. In some cases, patients may experience injection site and immune reactions which can be reduced by tolerizing the individual to the drug through the injection of low doses over a period of time (Okamoto et al., 1993). A two week protocol of less than 100 mcg/ml i.m. per day permits the immune system to acclimatize to the drug prior to initiating higher and chronic therapeutic dose schedules.

For topical applications, the applicable concentration of the present neurotoxin range from a minimum of 6 mcg per gram of a topical formulation up to 1 mg per gram. The applicable topical concentrations of venom are 2-3 fold greater than that for the purified neurotoxin as the neurotoxin accounts for approximately 40% of the composition of the venom. The average drug concentration of 20 mcg per gram of the topical preparation is preferable. The rate of application can range from an infrequent, as needed basis, to several applications per day particularly where the application is for the control of pain. The treatment of a condition like shingles may require 4 to 5 topical applications per day in order to reduce pain and speed healing.

In the following examples the analgesic effects of crotoxin were assessed using several animal models in addition to reported effects in humans.

### EXAMPLE 1

### Effects of Crotoxin on Pain Responses in Mice and Rats

Crotoxin 29.5, 44.3, or 66.5 g/kg (ip) exhibited a dose-dependent prolongation in the latency for the mouse to respond to the pair stimulation induced by heat. The analgesic effect of Crotoxin appeared at 1 h and peaked at 3 h following drug administration. The ED₅₀ of the antinociceptive effect of Crotoxin was 53.70 g/kg (42.55-67.77, 95% confidence limit) with hotplate test. Similarly, Crotoxin elicited a dose-dependent inhibition in the writhing response. The ED₅₀ of the antinociceptive effect of Crotoxin was 39.02 g/kg (28.59-53.20, 95% confidence limit) with acetic acid-writhing test. Crotoxin 44.3 g/kg also significantly inhibited the tail-flick reflex in rats. The analgesic effect of Crotoxin reached its' peak 2-3 h after drug administration in tail-flick test in rats.

### EXAMPLE 2

### Analgesic Actions of Centrally Administered Crotoxin

In mice, intra-cerebral ventricle (icv) administration of Crotoxin 0.3 g/kg ( 1/130 of the systemic dose of Crotoxin), significantly reduced the writhing response induced by acetic acid (p 0.05), indicating that icv injection of Crotoxin had marked analgesic effects. In the rat hotplate test, periaqueductal gray (PAG) administration of Crotoxin 0.15 g/kg ( 1/260 of systemic dose of Crotoxin) also produced a significant analgesic action (p 0.05). The analgesic effect of periaqueductal gray administered Crotoxin appeared 10 min following drug administration and lasted for about 90 min.

### EXAMPLE 3

### Effects of Atropine and Naloxone on Analgesia Induced by Crotoxin

In the hotplate test and acetic acid-writhing test in mice, Atropine alone at 0.5 mg/kg (im) or 10 mg/kg (ip) had no significant effect on the pain threshold. Both Crotoxin 44.3 g/kg and Crotoxin combined with Atropine exhibited significant analgesia (p 0.05). There was no significant difference between the two groups. In other experiments, Atropine at doses from 0.25-1 mg/kg in the hotplate test or 2.5-10 mg/kg in the acetic acid-writhing test, had no significant effect on Crotoxin-induced analgesia (data not shown). In the hotplate test, Naloxone at 3 mg/kg (ip) had no significant influence on the pain threshold. Both Crotoxin 44.3 g/kg and Crotoxin combined with Naloxone produced similar analgesic effects (p 0.05). There was no significant difference between these two groups. In other experiments, Naloxone at doses from 1.25-5 mg/kg in hotplate test had no effect or Crotoxin-induced analgesia

### EXAMPLE 4

### Effects of Acetylsalicylic Acid on Analgesia of Crotoxin in the Mouse Acetic Acid Writhing Test

The mice were randomly divided into 4 groups (n=10 in each group): saline control, crotoxin 44.3 g/kg⁻¹, acetylsalicylic acid (Ace) 300 mg/kg⁻¹, and crotoxin+acetylsalicylic acid. Acetylsalicylic acid 300 mg/kg⁻¹ (im) and crotoxin 44.3 g/kg⁻¹ (im) were administrated at the same time. Pain threshold was determined 2 h after crotoxin administration. In the mouse acetic acid test, acetylsalicylic acid 0.3 g.kg⁻¹ and crotoxin 44.3 g.kg⁻¹ produced significant analgesic effects. The analgesia of crotoxin combined with acetylsalicylic acid was stronger than that of either crotoxin or acetylsalicylic acid alone. There was a significant difference between two groups.

### EXAMPLE 5

### Human Subject with RA

A human male, aged 74 with rheumatic pain in his hands utilized crotoxin at 20 mg/g topical base incorporating penetration enhancers. Application was on an as needed basis. The patient observed a decrease in pain characterized as allowing him to feel comfortable. Along with the loss of pain was an increase in mobility in the areas to which the therapeutic was applied. The therapeutic produced a positive effective within 20 minutes and relief lasted approximately 4 hours.

The results clearly demonstrate that Crotoxin has analgesic effects. Furthermore the results show that crotoxin has analgesic effects in the absence of cancer or other chronic pain conditions. The results suggest that the site of analgesic actions of crotoxin may be mainly mediated by the central nervous system. The central cholinergic system and the central endogenous opioid peptidergic system appear not to be involved in antinociceptive actions of crotoxin. The combination of acetylsalicylic acid with crotoxin can increase the analgesic effects of crotoxin, suggesting that crotoxin may also produce peripheral analgesia similar to acetylsalicylic acid in addition to its central actions.

The data further suggested that the combination of the drugs could be beneficial giving the useful rapid onset style effectiveness displayed by opiates and the prolonged effect of the Crotoxin, in addition to the enhancement of the antinociceptive effect with Acetylsalicylic acid. Further benefits would be reduced dependence on a known addictive drug with significant side effects.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

What is claimed is:

1. A pharmaceutical composition comprising a therapeutically effective amount of toxin from the group including crotoxin and mojavetoxin having corresponding biological activity and a pharmaceutically acceptable carrier for use in inhibiting or controlling pain.

2. The composition of claim 1 wherein the crotoxin is obtained from the snake Crotalus durissus terrificus and the mojavetoxin is obtained from the snake Crotalus scutulatus scutulatus.

3. The composition of claim 1 which further comprises a therapeutically effective amount of acetylsalicylic acid whereby the toxin and acetylsalicylic acid together produce a synergistic effect providing enhanced pain relief.

4. The composition of claim 1 for parenteral (intravenous, intramuscular or subcutaneous) administration comprising between 10 mcg/kg and 3 mg/kg of toxin.

5. The composition of claim 1 for topical administration comprising substantially between 6 mcg and 1 mg of toxin per gram of base.

6. The composition of claim 5 in which the toxin is crotoxin at a concentration of 100-200 mcg per gram of base.

7. A method of producing and enhancing analgesia comprising administering an effective amount of toxin from the group including crotoxin and mojavetoxin having corresponding biological activity that is characterized by its ability to bind to presynaptic receptor sites resulting in an inhibition of aceylcholine release.

8. A method of treatment of pain in one of the human and the animal body comprising administering an effective amount of a composition comprising a toxin from the group including crotoxin and mojavetoxin having corresponding biological activity.

9. The method of claim 7 wherein the composition includes a therapeutically effective amount of acetylsalicylic acid whereby the toxin and acetylsalicylic acid together in composition produce a synergistic effect providing enhanced pain relief.

10. The method of claim 8 wherein the composition includes a therapeutically effective amount of acetylsalicylic acid whereby the toxin and acetylsalicylic acid together in composition produce a synergistic effect providing enhanced pain relief.

11. The method of claim 5 comprising administering the toxin composition ranging from at least once every other day to several applications daily.
